(19) United States
(12) Patent Application Publication
(10) Pub. No.: US 2007/0238188 A1
(43) Pub. Date: Oct. 11, 2007

(54) Peroxide monitor

(76) Inventor: Gregory E. Carr, Portland, OR (US)

Correspondence Address: TROP PRUNER & HU, PC, 1616 S. VOSS ROAD, SUITE 750, HOUSTON, TX, 77057-2631, US

(21) Appl. No.: 11/392,119

(22) Filed: Mar. 29, 2006

(51) Int. Cl. G01N 33/00; G01N 33/48

(52) U.S. Cl. 436/135; 422/68.1

(57) ABSTRACT

The presence and amount of hydrogen peroxide in a liquid can be determined by breaking down hydrogen peroxide into water and oxygen gas. By monitoring a base level of dissolved oxygen and comparing that to the amount of oxygen after breaking down the hydrogen peroxide, one can determine the amount of hydrogen peroxide in the liquid flow.

### BACKGROUND

This invention relates generally to monitoring the amount of hydrogen peroxide in liquids.

Hydrogen peroxide is present in some streams generated by various manufacturing facilities, including semiconductor manufacturing facilities. For example, hydrogen peroxide may be found in small amounts in pure water systems, including those that reclaim processed waste.

Measuring and controlling peroxide is of interest for a variety of reasons. Peroxide contamination of reclaimed streams can degrade oxygen removal processes, and damage ion exchange resins, filters, and degasifier membranes. Thus, it may be important to rapidly detect the presence of peroxide so that contaminated water can be diverted out of a reclaimed stream without damaging recovery processes.

Small amounts of hydrogen peroxide are continuously generated by ultraviolet light exposure used, for example, in sterilization or in organic destruction in ultra pure water systems. This peroxide has been implicated in the destruction of cartridge filter membranes and degasifier membranes.

### BRIEF DESCRIPTION OF THE DRAWINGS

FIG. 1 is a schematic depiction of one embodiment of the present invention;

FIG. 2 is a schematic depiction of another embodiment of the present invention;

FIG. 3 is a water system in accordance with one embodiment; and

FIG. 4 illustrates test results in accordance with one embodiment of the present invention.

### DETAILED DESCRIPTION

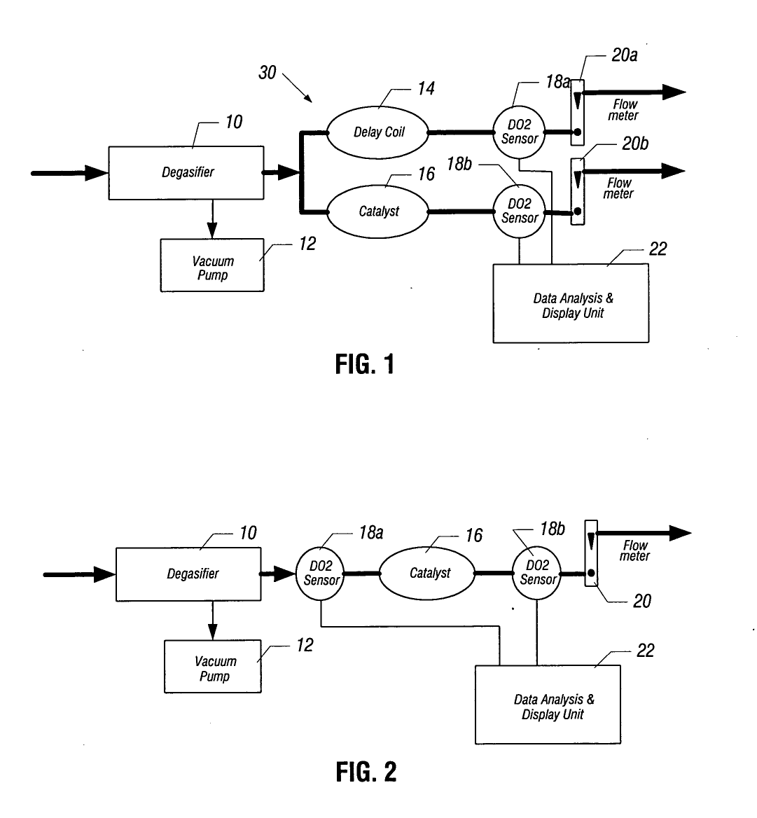

Referring to FIG. 1, a hydrogen peroxide detector 30 may receive a stream of liquid as indicated by the arrow on the left. The stream of liquid may be water and may be reclaimed or even pure water. To the right are two arrows indicating the output of the hydrogen peroxide detector 30. These arrows may proceed to various downstream systems which may, for example, include recovery components which may be adversely affected by the presence of hydrogen peroxide. Thus, in some cases, upon the detection of hydrogen peroxide, the detector 30 depicted in FIG. 1 can automatically cause the diversion of the flow for further processing, as well as the protection of downstream peroxide sensitive recovery components.

Still referring to FIG. 1, initially, the liquid flow enters a degasifier 10. In some embodiments, the degasifier 10 may be a commercially available device for removing entrained gasses from the liquid. As an example, the degasifier may be a membrane degasifier which is made of a tube (not shown) which is gas permeable. A vacuum is drawn on the outside of the tube by a vacuum pump 12. When the incoming liquid circulates through the tube, the gas flows outwardly through the tube lumen and is collected by the vacuum pump 12. The liquid remains and continues on.

Next, in the embodiment shown in FIG. 1, the liquid flow is split into two equal flows. One flow proceeds through a catalyst containing unit 16. In one embodiment, the catalyst containing unit may be a tank, tube, or container having a peroxide destruction catalyst which decomposes hydrogen peroxide into water and oxygen gas. In one embodiment, the catalyst may be a pelletized mixture of copper and manganese oxide. In another embodiment, the catalyst may be a platinum catalyst. However, those skilled in the art are aware of a wide array of catalysts which may be utilized to break down hydrogen peroxide.

After the hydrogen peroxide has been broken down into water and oxygen gas, a sensor 18b senses dissolved oxygen in the continuing liquid flow. Since the liquid has already been degassed by the degasifier 10, the dissolved oxygen is proportional to the amount of hydrogen peroxide broken down by the catalyst.

Thereafter, the flow continues through a flow meter 20b. The flow meter 20b may be utilized to control the flow of liquid through the catalyst containing unit 16 to ensure that the catalyst is being used at an efficient liquid flow rate.

The dissolved oxygen sensor 18b may be coupled to a data analysis and display unit 22. This unit 22 may be a programmable system which analyzes the data from the sensor 18b.

At the same time and in parallel, the second liquid flow path passes through a delay coil 14. The delay coil 14 may include a circuitous route which may match the delay of the circuitous route which the liquid takes in passing through the catalyst containing unit 16.

Thus, the two flows may flow at substantially the same flow rate. This can be checked by the flow meters 20a and 20b. The flow rates of the flow meters 20a and 20b may be compared to the unit 22 to appropriately regulate and match the flows, if necessary, to one another to improve the action of the catalyst and to maintain the flow rates as constant as possible.

The flow through the delay coil 14 is subjected to dissolved oxygen sensing at the sensor 18a. Any sensed oxygen in the stream is an indication of the level of oxygen which was passed by the degasifier 10. The information obtained from the sensor 18a provides a baseline that may be subtracted from the dissolved oxygen sensed by the sensor 18b to determine the amount of hydrogen peroxide. The amount of peroxide is proportional to the amount of detected oxygen and may be determined using conventional addition methods. This analysis may be done by a programmable machine provided within the data analysis and display unit 22. The result may be displayed by the unit 22.

In accordance with another embodiment of the present invention, shown in FIG. 2, the degasifier 10 and vacuum pump 12 may operate as already described. Similarly, the dissolved oxygen sensors 18a, the catalyst containing unit 16, and the dissolved oxygen sensor 18b, as well as the flow meter 20, may all be as described previously.

However, in this embodiment, it is not necessary to split the liquid flow into two separate but parallel paths. Instead, the dissolved oxygen sensor 18a senses the amount of oxygen in the flow path before breakdown of the hydrogen peroxide into water and oxygen gas in the catalyst containing unit 16. The dissolved oxygen sensor 18b then senses how much additional oxygen results from the breakdown of hydrogen peroxide by the catalyst.

By subtracting the measured oxygen contents from the two oxygen sensors 18a and 18b, in the data analysis and display unit 22, the unit 22 can automatically determine the dissolved oxygen content from the peroxide breakdown. From this data, the unit 22 may thereby automatically derive the hydrogen peroxide content over time. Again, the flow meter 20 may be used to ensure that the flow through the catalyst containing unit 16 is in accordance with the effective operation of the catalyst.

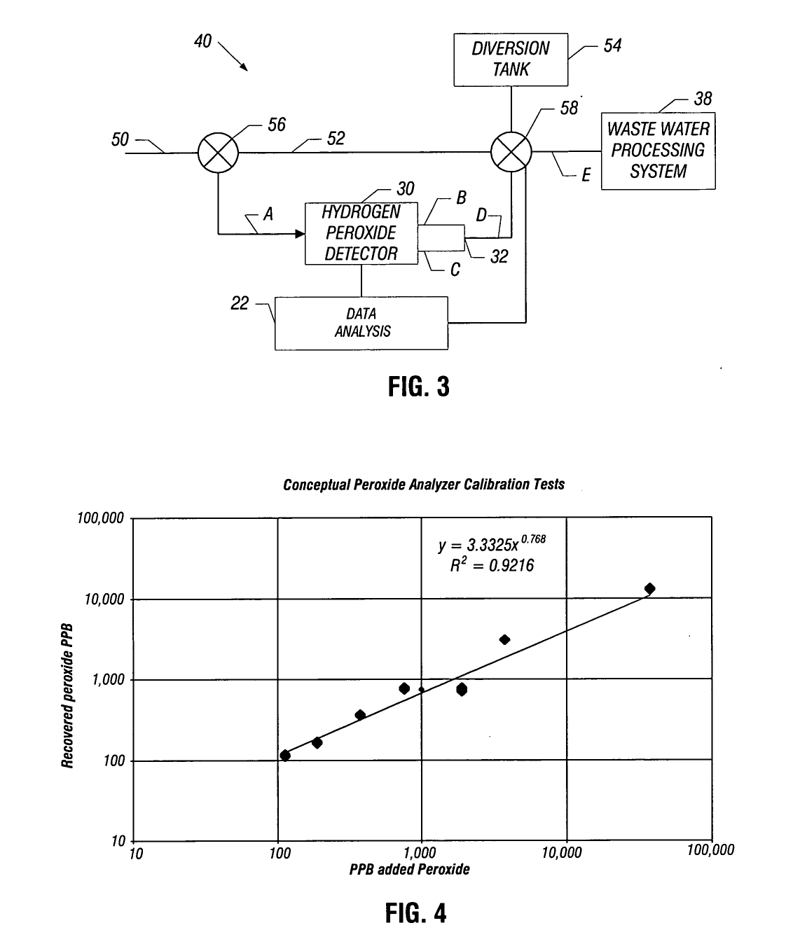

Referring to FIG. 3, a liquid treatment system, such as a wastewater treatment system 40, may utilize the peroxide detector 30 shown in FIG. 1 or, in an alternative embodiment, the peroxide detector 30 shown in FIG. 2. The liquid stream 50 passes through a sampling valve 56. The valve 56 may pass a small portion A of the stream 50 to the detector 30. The remainder 52 of the stream 50 proceeds to the valve 58.

An input flow A to the detector 30 is split into two flows at the output as indicated at B and C. These flows may be recombined at 32 to produce the combined flow D.

The combined flow D then enters a valve 58. The output of the valve 58 is the flow E which may go on to a conventional wastewater processing system 38. That wastewater processing system 38 may include components which would be sensitive to hydrogen peroxide, including, as examples, cartridge filters, membranes, oxygen removal processes, ion exchange resins, filters, and degasifier membranes.

If the data analysis unit 22 determines that the flow is peroxide rich, the flow may be diverted by the valve 58 to a diversion tank 54 for further processing. In other words, if the peroxide content is too high, instead of relying on the detector 30 to remove the peroxide, the flows 52 and D may be diverted for further processing in some embodiments. In this way, wastewater processing system 38, coupled to the flow path E, may be protected from extremely high amounts of hydrogen peroxide.

Referring to FIG. 4, the recovered oxygen, in parts per billion, is graphed versus the parts per billion of added peroxide in a test setup. As the graph shows, the systems depicted in FIGS. 1 and 2 may be sensitive to less than ten parts per billion of peroxide.

Some embodiments of the present invention may be advantageous because the measurements are essentially continuous and instantaneous, chemical reagents may not be needed, and the analyzer may be relatively inexpensive. Moreover, the detection limits may be in the tens of parts per billion in some embodiments.

References throughout this specification to “one embodiment” or “an embodiment” mean that a particular feature, structure, or characteristic described in connection with the embodiment is included in at least one implementation encompassed within the present invention. Thus, appearances of the phrase “one embodiment” or “in an embodiment” are not necessarily referring to the same embodiment. Furthermore, the particular features, structures, or characteristics may be instituted in other suitable forms other than the particular embodiment illustrated and all such forms may be encompassed within the claims of the present application.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A method comprising:
breaking down hydrogen peroxide into water and oxygen to detect hydrogen peroxide in a liquid.

2. The method of claim 1 including degasifying the liquid before breaking down the hydrogen peroxide.

3. The method of claim 2 including detecting dissolved oxygen after degasifying the liquid.

4. The method of claim 1 including detecting hydrogen peroxide in a flowing liquid.

5. The method of claim 4 including splitting a degasified liquid stream into two flows.

6. The method of claim 5 including using a catalyst to break down hydrogen peroxide in one flow.

7. The method of claim 6 including sensing the amount of oxygen in the one flow after breaking down hydrogen peroxide.

8. The method of claim 7 including sensing the amount of dissolved oxygen in the other flow.

9. The method of claim 8 including delaying the second flow to match the flow rate through the catalyst.

10. The method of claim 9 including comparing the dissolved oxygen in the two flows to determine the amount of peroxide in the liquid.

11. The method of claim 1 including degasifying the liquid, sensing the amount of dissolved oxygen, breaking down hydrogen peroxide after sensing the amount of dissolved oxygen, and then sensing the amount of dissolved oxygen after breaking down hydrogen peroxide.

12. The method of claim 1 including continuously measuring the amount of dissolved hydrogen peroxide in a flowing liquid.

13. The method of claim 1 including monitoring the flow through a catalyst breaking down hydrogen peroxide into water and oxygen gas.

14. The method of claim 13 including providing a first flow through the catalyst and a second flow which bypasses the catalyst and monitoring the flow rates of each of said flows.

15. A peroxide detector comprising:
a catalyst containing unit to breakdown hydrogen peroxide in a liquid; and
a dissolved oxygen sensor to sense dissolved oxygen in the liquid after exposure to the catalyst.

16. The detector of claim 15 wherein said detector including a liquid flow pathway, said unit and said sensor being arranged along said pathway.

17. The detector of claim 15 including a degasifier to remove gas from a liquid before said liquid is exposed to the catalyst.

18. The detector of claim 15 including a pair of oxygen sensors.

19. The detector of claim 18 including first and second liquid passageways each including a sensor, said first liquid passageway including said unit.

20. The detector of claim 19 wherein said second liquid passageway includes a delay device to delay a liquid flow.

21. The detector of claim 20 wherein said delay device delays a liquid flow through the second passageway to cause delay times in said passageways to be substantially equal.

22. The detector of claim 20 including a flow meter in each passageway.

23. The detector of claim 18 including a flow passageway through said catalyst unit, said sensors being arranged along said passageway on opposed sides of said catalyst containing unit.

24. A liquid treatment system comprising:
a liquid treatment device; and
a hydrogen peroxide detector coupled to said device, said detector including a catalyst to breakdown hydrogen peroxide in a liquid.

25. The system of claim 24 including a control to control the flow of liquid to said device based on the amount of hydrogen peroxide in said flow.

26. The system of claim 24 wherein said control to automatically control the flow of liquid to said device based on the amount of hydrogen peroxide in said flow detected by said detector.

27. The system of claim 26 wherein said control includes a valve and a data analysis unit to control said valve.

28. The detector of claim 24 including a degasifier to remove gas from a liquid before said liquid is exposed to the catalyst.

29. The system of claim 24 wherein said detector to detect the amount of oxygen in the flow after the hydrogen peroxide is broken down by said catalyst.

30. The system of claim 29 including a device to convert the amount of oxygen in the flow to a measure of the hydrogen peroxide in the flow before exposure to the catalyst.
